SEQUENCE LISTING (1 sequence)

SEQ ID NO: 1 - DNA, 185 bp, H1N1 swine influenza virus
ggtgctataaacaccagcctgatggtgataaccgtaccggacattytccaattgtgttgccggtttcattgaaggctgtrgccagtctcaattttgtgttttctgaagtyccatttcagaatatacatccratcccgtctattcaatctagaggcttttctgaagatccatctaccatccctgtc

(19) United States
(12) Patent Application Publication
(10) Pub. No.: US 2012/0329058 A1
(43) Pub. Date: Dec. 27, 2012

(54) METHOD AND DEVICE FOR FAST DETECTING NUCLEIC ACID

(75) Inventors: Guohua Zhou, Nanjing (CN); Chao Liang, Nanjing (CN); Chuanjun Li, Nanjing (CN)

(73) Assignee: HUADONG RESEARC INSTITUTE FOR MEDICINE AND BIOTECHNICS, Nanjing, Jiangsu (CN)

(21) Appl. No.: 13/520,116

(22) PCT Filed: Dec. 23, 2010
(86) PCT No.: PCT/CN2010/080176

§ 371 Date: Aug. 29, 2012

(30) Foreign Application Priority Data
Dec. 30, 2009 (CN) 200910264174.8

(51) Int. Cl. G01N 21/64; C12M 1/42; C12M 1/38; C12M 1/34

(52) U.S. Cl. 435/6.12; 435/287.2; 435/286.1

(57) ABSTRACT

The invention belongs to the field of medical detection and discloses a method and device for fast detecting nucleic acid. The pathogenic microorganism nucleic acid is carried out with amplification reaction by LAMP technology in a temperature-controlled reaction tube comprising at least a sealing layer, and after the reaction is completed, the temperature of the reaction tube is raised without opening the tube to dissolve the sealing layer to release the fluorescent dye for the fluorescence detection. The method can carry out the amplification reaction and fluorescence detection directly in the instrument without taking out the reaction tube. The device has advantages of simple structure, low cost and simple operation, and can be used as a fast detecting device for the pathogenic microorganism nucleic acid.

### FIELD OF THE INVENTION

The invention relates to a field of medical detection and more particularly relates a method and device for fast detecting nucleic acid.

### BACKGROUND OF THE INVENTION

In recent years, various deadly infectious diseases spread widely worldwide and caused huge economic losses to human beings as well as hardly threatened the lives and health of human beings, such as SARS, bird flu H5N1, Streptococcus suis, influenza H1N1. The methods of detecting pathogenic microorganisms mainly include morphological observation with microscopes, immunization detection, nucleic acid detection and so on.

Compared with other methods of detecting pathogenic microorganisms, the nucleic acid detection has advantages of high sensitivity, good specificity, short window period, short detecting time and so on. The nucleic acid amplification technology has been widely used for clinic detecting the pathogenic microorganisms. At present methods of detecting the pathogenic microorganism nucleic acid mainly comprise polymerase chain reaction (PCR), rolling circle amplification (RCA), loop-mediated isothermal amplification (LAMP) and so on.

PCR technology is the most common one in the existing nucleic acid detecting technologies, wherein the Real-time PCR technology is the most common one. This technology has advantages of high sensitivity, good specificity and so on. However an expensive Real-time PCR instrument is needed, and the price is about several hundreds of thousands Yuan RMB. Such an expensive cost limits the wide application thereof in clinics.

The main technical requirement of the RCA technology and LAMP technology is isothermal amplification so an expensive PCR instrument is unnecessary, and it can be instead by a constant temperature heater, as a constant temperature water bath. As one of the nucleic acid amplification technologies, compared with traditional PCR technology, the LAMP has an important advantage of high sensitivity, and its reaction is carried out at constant temperature, thereby, an expensive PCR instrument is unnecessary. At present detections for products of RCA and LAMP are carried out by opening the tube when the reaction was finished. In this way the step of opening tube is not only increasing one operation step but also causing lag pollution of the amplified products.

Although, the technology of detecting the pathogenic microorganism with nucleic acid has been widely applied in clinics, the above-mentioned disadvantages are still existed.

### SUMMARY OF THE INVENTION

The purpose of the invention is to provide a method for fast detecting nucleic acid which can avoid lag pollution of the amplified product, has high sensitivity and low cost.

Another purpose of the invention is to provide a detecting device used in above-mentioned detecting method.

The inventor has discovered from his research work: 1. LAMP technology can amplify the pathogenic microorganism nucleic acid sample, including DNA and RNA, and has a high sensitivity; 2. SYBR Green 1 dye can be embedded into the internal part of the nucleic acid double-chain structure, and produce fluorescence under the exciting light. Based on these, conceptions of the present invention made by the inventor are as follows:

A method for fast detecting nucleic acid, according to the method, a pathogenic microorganism nucleic acid is carried out with an amplification reaction by LAMP technology in a temperature-controlled reaction tube comprising at least a sealing layer, and after the amplification reaction is finished the temperature of the reaction tube is raised without opening the tube to dissolve the sealing layer in which a fluorescent dye is sealed therein and the fluorescent dye is released for the fluorescence detection.

According to the detecting method, wherein the temperature-controlled reaction tube comprises two sealing layers, all or part of the reagents used in the nucleic acid amplification reaction are sealed in the first sealing layer which is melted at a lower temperature, the fluorescent dye is sealed in the second sealing layer which is melted at a higher temperature, the sequent dissolutions of two sealing layers are realized by controlling the change of temperatures, so as to control the process for the nucleic acid amplification reaction and fluorescence visual inspection.

According to the detecting method, wherein said fluorescent dye is SYBR Green 1 dye.

According to the detecting method, wherein the amplification reaction and fluorescence detection are carried out directly in the sealed temperature--controlled reaction tube.

According to said detecting method, wherein the fluorescence detection is realized by visual inspecting or photographing or a photoelectric sensor collecting and analyzing images or data of the fluorescence issued by the fluorescent dye under the exciting light.

A detecting device used in the detecting method comprises a reaction tube oscillating device, a temperature adjusting device, a time adjusting device, a fluorescence color observing device, and a temperature-controlled reaction tube with at least a sealing layer which melts at a proper temperature, each device is respectively connected with a central control circuit: the sealing layer seals all or parts of reagents needed in the reaction process according to the characteristics of the reaction process to be controlled. A reaction tube lifting device connected with the central circuit is further included.

According to said detecting device, wherein the reaction tube oscillating device comprises a reaction tube rack, a rack oscillating motor which is connected with the reaction tube rack; the reaction tube lifting device comprises a drive motor for lifting the reaction tube rack and a reaction tube rack lifting rod, the drive motor for lifting the reaction tube rack is connected with the reaction tube rack lifting rod, and the reaction tube rack lifting rod is connected with the reaction tube rack.

According to said detecting device, wherein the temperature control device comprises a temperature control module, a heater and a temperature measuring device; the heater and the temperature measuring device are respectively connected with the temperature control module, the temperature control module is connected with the central control circuit or directly forms one part of the central control circuit, the temperature sensor of the temperature measuring device is near to the lower part of the reaction tube or near to the heater; the temperature adjusting device also can comprise a radiator connected with the temperature control module.

According to said detecting device, wherein the fluorescence color observing device comprises a fluorescence exciting light source and a fluorescence color observing or collecting device; the fluorescence color observing device is an observing window with or without a filter; the fluorescence color collecting device is an image collecting device or a photovoltaic conversion data collecting and analyzing device; the fluorescence exciting light source gives out light and irradiates the reaction solution in the reaction tube, and the fluorescence color observing or collecting device can observe or collect the fluorescence signal from the reaction solution in the reaction tube.

According to said detecting device, wherein the central control circuit further comprises a control information input or stored program control reaction type selecting device.

In order to save the cost of the instrument, the fluorescence color observing device preferably adopts an observing window, and adjusting of temperature, time, oscillating and so on preferably adopt a way of presetting program controlled reaction type in the central control circuit.

The wavelength of the excitation light source and the wavelength filtered by the filter are determined according to the wavelength of the excitation light source of the fluorescent dye and the wavelength of the emitted fluorescence. The invention relates to an SYBR Green I fluorescent dye, wherein this fluorescent dye is excited at 497 nm, and the wavelength of the emitted fluorescence is 520 nm.

The temperature-controlled reaction tube adopted in the invention comprises at least a sealing layer, the reagents needed in the reaction process which needs to be controlled are sealed in the sealing layer, the melting of the sealing material is realized by controlling the temperature to release the sealed reagent so as to control the reaction process.

When multiple sealing layers are provided, the sealing layers are installed at the position where the reagents in the sealing layers contact with the reaction system (usually reaction solution) of the former reaction process after the sealing layers are melt (actually the sealing materials forming the sealing layers are melt; in order to bring convenience to description, “sealing layers are melt” is adopted, similarly hereinafter) according to the sequences of different reaction processes; the melting temperature of each sealing layer is less than or equal to the reaction temperature of the corresponding reaction process, and is higher than the reaction temperature of the former reaction process, and is lower than the melting temperature of the corresponding sealing layer of the later reaction process (if the sealing layer is adopted in the former reaction process, whether the melting temperature of the sealing layer is higher than the reaction temperature of the former reaction process is not taken into account as no former reaction process exists; in the same way, whether the melting temperature of the sealing layer is lower than the melting temperature of the corresponding sealing layer in the later reaction process is not taken into account as no corresponding sealing layer in the later reaction process exists). The melting order of each sealing layer is arranged from low to high according to the reaction temperatures.

Said reaction tube is arranged with sealing layers in sequence according to the order of different reaction processed which need to be controlled by the sealing layers, the outermost sealing layer (namely the sealing layer closest to the reaction tube mouth) corresponds to the reaction process to be performed earlier.

According to said reaction tube, the temperature of said reaction tube is sectional adjusted according to the reaction process which needs to be controlled, is higher than or equal to the reaction temperature of the corresponding reaction process and lower than the melting temperature of the sealing layer corresponding to the later reaction process. Preferably the temperature of the reaction tube is adjusted to the reaction temperature needed in corresponding reaction process, as the melting point is less than or equal to the reaction temperature, the sealing layer corresponding to the reaction process is melt to release the reagent sealed in the sealing layer so that the corresponding reaction process can be performed.

According to said reaction tube, the temperature of the reaction tube is adjusted from lower temperature to high temperature according to the sequence of the reaction processes.

According to said reaction tube, different sealing layers adopt materials with different melting points as sealing materials. The materials with different melting points are paraffins with different melting points or low melting point PTFE with different melting points.

According to said reaction tube, the quantity of said sealing layer is designed according to the quantity of the reaction phases; the melting points of the sealing layers corresponding to the reaction processes at different temperature are different.

According to said reaction tube, wherein the reagent in the sealing layer is mixed in the sealing material or separated by the sealing material.

When preparing a temperature-controlled reaction tube, firstly reagent needed in corresponding reaction phase is added into the tube, then proper paraffins is added on the surface of the reagent (taking paraffins as an example, also other sealing layer materials can be used), the paraffins is heated to melt, and cooled to the room temperature, then actually reagent is sealed by the paraffins. According to the quantity of different processes which need sealing layers to control in the whole reaction, the quantity of the sealing layers in the reaction tube designed according to temperature and the sequence (or position), paraffin with a proper melting point is chosen as the sealing layer material according to the temperatures of different reaction processes.

As the invention mainly relates to an isothermal amplification LAMP technology and fluorescence detection reaction, the actually used temperature-controlled reaction tube only need to be provided with one or two sealing layers.

### Advantages of the Invention

The fast detecting device provided in the invention can combine the nucleic acid isothermal amplification technology with the fluorescence technology so as to detect the nucleic acid amplification product via the fluorescence after the reaction is finished. The biggest advantage of the instrument is that after the reagent preparation is finished, the reaction can be performed after putting into the instrument and the fluorescence detection can be performed automatically after the nucleic acid amplification reaction is finished without taking-out the reaction tube and opening the reaction tube, therefore the operation steps are reduced as well as the lag pollution of the reaction product is avoided at the utmost. The invention reduces the cost of the reaction instrument, also realizes the combination of nucleic acid amplification reaction and the detecting reaction, overcomes the lag pollution of the nucleic acid amplification product, and also has advantages of simple structure, convenient for carrying, low cost, convenient for operating, fast reaction and so on, and is suitable for being used as a clinic or outdoor on-field fast detecting device.

The invention also provides a reaction tube which can control the reaction process via temperature changes, release the regent sealed in the reaction tube so as to control the processes such as reaction starting, terminating and detecting. This technology effectively avoids the template pollution caused by frequently opening the reaction tube before the reaction and lag pollution caused by opening the reaction tube when the reaction is finished, also controls the starting time of the reaction to some extent, and enhances the specificity of the reaction. This reaction tube can be widely used on basic research of biomedicine filed and fields of biological analysis, pathogenic microorganism inspection and disease diagnosis.

### BRIEF DESCRIPTION OF THE DRAWINGS

FIG. 1 is a structure schematic diagram of the detecting device. Wherein: 1. Power and central control circuit; 2. Time adjusting device (timing module); 3. Temperature control module; 4. Heater; 5. Rack oscillating device; 6. Reaction tube rack; 7. Observing window; 8. Sample entrance; 9. Temperature-controlled reaction tube; 10. Excitation light source; 17. Drive motor for lifting reaction tube rack; 19. Radiator; 20. Temperature measuring device.

FIG. 2 is a schematic diagram of the detecting device. Wherein: 1. Power and control circuit; 2. Timing module; 3. Temperature control module; 4. Heater; 5. Rack oscillating device; 6. Reaction tube rack; 10. Excitation light source; 19. Radiator; 20. Temperature measuring device.

FIG. 3 is an appearance schematic diagram of the detecting device. Wherein: 7. Observing window; 8. Sample entrance.

FIG. 4 is a schematic diagram of the excitation light source. Wherein: 9. Temperature-controlled reaction tube; 10. Excitation light source.

FIG. 5 is a schematic diagram of lifting and oscillating device. Wherein: 4. Heater; 5. Oscillating motor; 6. Reaction tube rack; 9. Temperature-controlled reaction tube; 10. Excitation light source; 17. Drive motor for lifting reaction tube rack; 18. Reaction tube rack lifting rod.

FIG. 6 is a schematic diagram of the temperature-controlled reaction tube. Wherein: 11. Sealing material 1; 12. Sealing material 2; 13. Reagent 1; 14. Reagent 2; 15. Tube body; 16. Tube cap. The sealing material 1 and reagent 1 form the sealing layer 1, the sealing material 2 and the reagent 2 form the sealing layer 2.

FIG. 7 is a schematic diagram of sealing layers at different temperatures of the temperature-controlled reaction tube of the invention.

### DETAIL DESCRIPTION OF THE INVENTION

The invention is further described in detail by the following embodiments.

### EXAMPLE 1

### Construction of the Detecting Device and Work Process

1. Construction

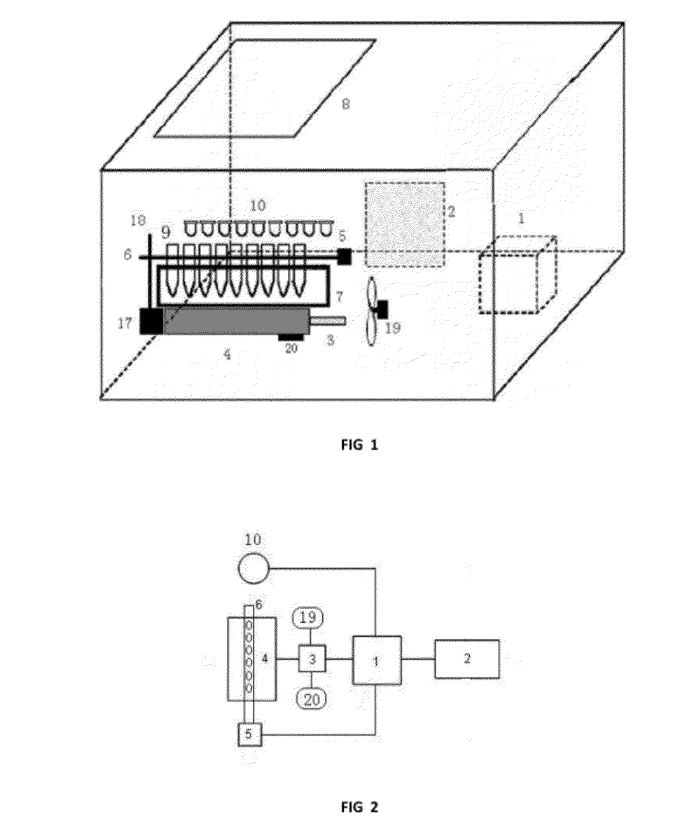

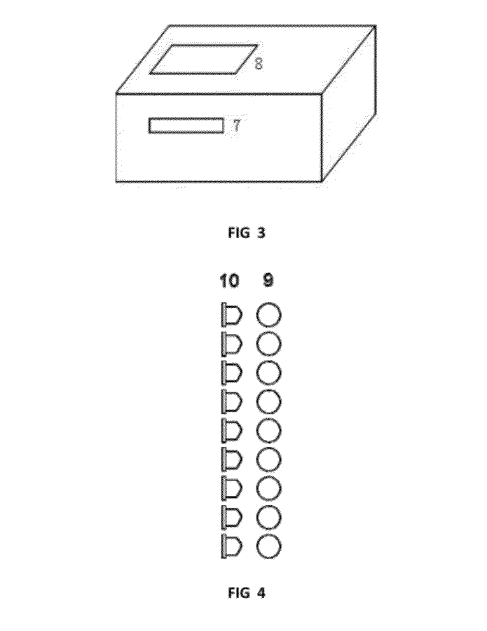

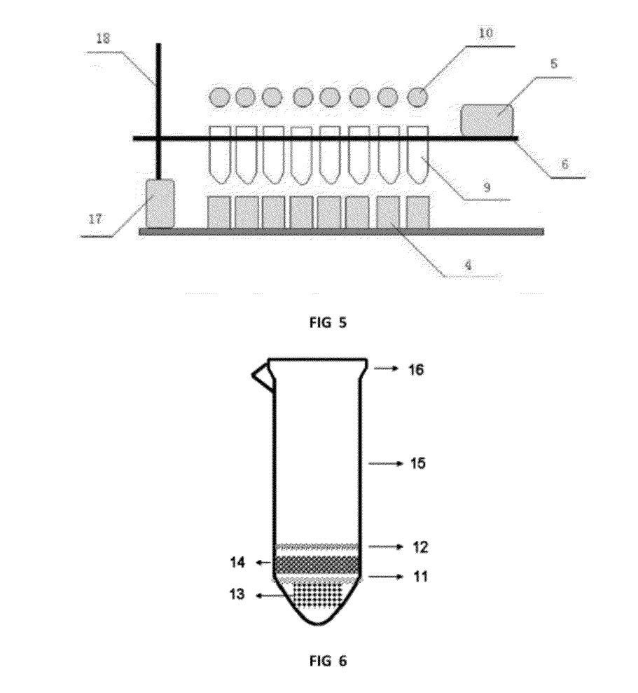

Referring to FIG. 1˜5, the detecting device comprises a reaction tube oscillating device, a temperature adjusting device, a time adjusting device 2 (timing module 2) and a fluorescence color observing device which are connected with a central control circuit 1; also comprises a reaction tube lifting device connected with the central control circuit 1; (also can comprise a temperature-controlled reaction tube 9, the detail of the temperature-controlled reaction tube will be described in the Example 2).

The reaction tube oscillating device comprises a reaction tube rack 6 and a rack oscillating motor (for example a cam motor) 5, the rack oscillating motor is connected with the reaction tube rack. the reaction tube lifting device comprises a drive motor for lifting the reaction tube rack 17 and a reaction tube rack lifting rod 18, the drive motor for lifting the reaction tube rack is connected with the reaction tube rack lifting rod, and the reaction tube rack lifting rod is connected with the reaction tube rack. The reaction tube oscillating device can oscillate the reaction tube in the reaction process to mix the reaction systems and promote the reaction process; the reaction tube rack lifting device can bring convenience to pick and place the reaction tube, or put the reaction tube close to or far away from the heater. The temperature adjusting device comprises a temperature control module 3, a heater 4 and a temperature measuring device 20; the heater and the temperature measuring device are respectively connected with the temperature control module, the temperature control module is connected with the central control circuit or directly forms one part of the central control circuit; the temperature sensor of the temperature measuring device is located in the lower part of the reaction tube or is near to the heater (when the reaction tube is near to or is placed on the heater, the temperature of the heater is approximately equal to the temperature of the reaction solution of the reaction tube); the heater can be located below the reaction tube rack, or be placed in other parts of the instrument which adopt methods such as blowing hot air to the area of the reaction tube (when the reaction tube is filled with much solution, directly heating can cause the local high temperature of the bottom part of the reaction tube, which can be avoided by the above-mentioned method of blowing hot air to the area of the reaction tube, so as to keep a uniform temperature of the reaction solution); the temperature adjusting device also can comprise a radiator 19 connected with the temperature control module.

The fluorescence color observing device comprises a fluorescence excitation light source 10 and a fluorescence color observing or collecting device; the fluorescence color observing device is an observing window 7 with or without a filter; the fluorescence color collecting device is an image collecting device or a photoelectric conversion data collecting and analyzing device; the fluorescence excitation light source 10 irradiates the reaction solution in the reaction tube 9, and the fluorescence color observing or collecting device can observe or collect the fluorescence signal emitted from the reaction solution in the reaction tube. In order to save the cost of the instrument, preferably the observing window way is adopted.

The central control circuit also can comprise a control information input device. The time setting device and temperature setting device also can be integrated into the control information input device, other information for example whether an oscillating reaction tube is needed in the reaction process, the oscillating time and frequency of the reaction tube, whether the reaction tube rack needs to be lifted can be input into the central control circuit from the control information input device, or directly be pre-programmed into the program of the central control circuit. Information such as time adjusting, temperature adjusting, oscillation adjusting for controlling the reaction process can be pre-programmed into the program of the central control circuit, the type of the corresponding program-controlled reaction can be chosen on the control information input device. The central control circuit adjusts the work of corresponding device theater, radiator, reaction tube rack lifting or oscillating device, excitation light source and so on) after receiving the control information and feedback information.

2. Work Process

1). (Power on) The temperature-controlled reaction tube 9 filled with reaction system is placed into the instrument via the sample entrance 8, and is placed on the reaction tube rack 6 (If a reaction tube rack lifting device is provided, it is convenient to pick and place the reaction tube).

2). The reaction time of corresponding reaction process is set via the timing module 2, the reaction temperature of corresponding reaction process is set via the temperature control module 3, the relative control information is controlled and adjusted by the central control circuit 1 (or choose the program reaction type pre-programmed into the central control circuit). After the setting is finished, the heater 4 begins to work and heats the reaction system to the set temperature, then stops heating after the temperature sensor of the temperature measuring device 20 gives feedback (if the temperature is too high, the radiator 19 can be started), then the nucleic acid amplification begins (whether an oscillating reaction tube is needed is determined according to the reaction).

3). After the nucleic acid amplification reaction is finished, the temperature is set to the temperature needed for melting the sealing layer by setting the temperature control module 3 (or adjusting by the program-controlled reaction type preset by the central control circuit), then the heater works to raise the temperature to the set temperature so as to melt the sealing layer to release the fluorescent dye in the reaction tube. The fluorescent dye is mixed with the reaction product via the oscillating device 5.

4). The excitation light source 10 is started to irradiate the reaction tube, the fluorescence produce situation is observed via the observing window 7 or the fluorescence information is collected and analyzed by photographing or via a photoelectric sensor and so on.

### EXAMPLE 2

### Temperature-Controlled Reaction Tube

Paraffin Supplier: Nan Yang Paraffin Fine Chemicals Factory

### Specifications and Characteristics of Paraffin

| Paraffin Model | Melting Point ( ) |
|---|---|
| 52# | 52-54 |
| 54# | 54-56 |
| 56# | 56-58 |
| 58# | 58-60 |
| 60# | 60-62 |
| 62# | 62-64 |
| 64# | 64-66 |
| 70# | 67-72 |
| 75# | 72-77 |
| 80# | 77-82 |
| 85# | 82-87 |
| 90# | 87-92 |
| 95# | 92-97 |

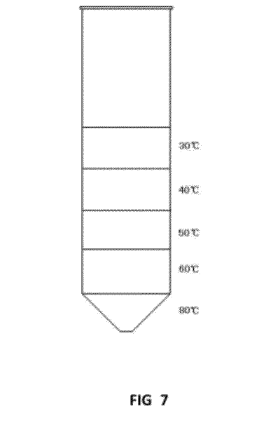

Referring to FIG. 7, the sealing layer is installed at the position where after the sealing layer is melted the released reagents in the sealing layer contact with the reaction system (usually it is reaction solution) of the former reaction process (for the first reaction, as no former reaction process exists, usually the reaction system is the object to be detected and other reagent needed in the first reaction excluding the sealed regent). The melting temperature of each sealing layer is less than or equal to the reaction temperature of the corresponding reaction process: and is higher than the reaction temperature of the former reaction process, (for the sealing layer of the first reaction, this condition is not taken into account, for example the 30° C. sealing layer in the FIG. 7); and is lower than the melting temperature of the corresponding sealing layer in the later reaction process (for the last melted sealing layer, this condition is not taken into account, for example the 80° C. sealing layer in the FIG. 7). The reagents in each sealing layer are respectively mixed in the sealing materials forming the scaling layers corresponding to each reaction process or are sealed by the sealing material. In reaction, the temperature of the reaction tube is staggered adjusted according to the reaction process controlled. The temperature is controlled higher or equal to the reaction temperature of the corresponding reaction process and lower than the melting temperature of the sealing layer for the later reaction process. (In order to avoid the disadvantageous influences to some reaction processes caused by the temperature of the reaction tube being higher than the reaction temperature of the corresponding reaction process, in this example the temperature of the reaction tube is adjusted to the needed reaction temperature in the corresponding reaction process.). Please refer to FIG. 7, the reaction temperatures of each reaction process in FIG. 7 are sequentially 30° C., 40° C., 50° C., 60° C., 70° C., 80° C., the melting temperatures of the sealing materials (for example paraffin) forming the sealing layers corresponding to each reaction process are respectively 28˜30° C., 38˜40° C., 48˜50° C., 58˜60° C., 68˜70° C., 78˜80° C., the initial reaction temperature is 30° C. The object to be detected is added, when the temperature of the reaction tube is raised to 30° C., the 30° C. sealing layer is melted to release the reagent needed in the first phase, then the first phase reaction is carried out, the melted paraffin floats on the top part of the reaction solution. After proper time of the reaction, the temperature of the reaction tube is raised to 40° C., the 40° C. sealing layer melts to release the reagent needed in the second phase, then the second phase reaction is carried out, and the melted paraffin floats on the top part of the reaction solution. After proper time of the reaction, the temperature of the reaction tube is raised to 50° C., the 50° C. sealing layer melts to release the reagent needed in the third phase, the third phase reaction is performed, and the melted paraffin floats on the top part of the reaction solution. By parity of reasoning, the fourth phase reaction is performed at 60° C., and the fifth phase reaction is performed at 80° C. After the reaction is finished, the temperature is reduced (or cooled), all or part of the paraffins floating on the top part of the reaction solution solidifies and seals the reaction solution to avoid lag pollutions.

### EXAMPLE 3

Six primers are designed in this example according to the HA gene of Swine Flu H1N1, and the LAMP amplification system is as follows:

TABLE 1
H1N1 LAMP Primer

| | |
|---|---|
| SW H1-F3: | 5′-GGTGCTATAAACACCAGCC-3′ |
| SW H1-B3: | 5′-TGATGGTGATAACCGTACC-3′ |
| SW H1-LF: | 5′-GGACATTYTCCAATTGTG-3′ |
| SW H1-LB: | 5′-TTGCCGGTTTCATTGAAGG-3′ |
| SW H1-FIP | 5′-CTGTRGCCAGTCTCAATTTTGTGttttCTGAAGTY |
| (Flc + F2): | CCATTTCAGAATATACATCCR-3′ |
| SW H1-BIP | 5′-ATCCCGTCTATTCAATCTAGAGGCttttCTGAAGAT |
| (Blc + B2): | CCATCTACCATCCCTGTC-3′ |

Y: t/u or c; R: g or a.

25 μL LAMP reaction system

| | |
|---|---|
| Buffer | 1.875 μL |
| BIP, FIP | 2.31 μL for each |
| B3, F3 | 0.19 μL for each |
| LB, LF | 1 μL for each |
| Bst DNA polymerase | 0.5 μL |
| Fluorescent dye | 0.5 μL |
| H₂O | 1.625 μL |
| Mineral oil | 8 μL |
| H1N1 template | 1 μL |

LAMP reaction procedure: 63° C./1.5 h→80° C./5 min

The temperature-controlled reaction tube is provided with a sealing layer which is sealed with fluorescent dye (for example SYBR Green I) by paraffins in advance (for example 90#). After the reaction tube is added with nucleic acid amplification system and samples to be detected, the reaction tube is put into the detecting device of the invention, the reaction system is heated to the temperature needed in the reaction to react with the samples for a corresponding period of time according to LAMP reaction procedure, after the reaction is finished, the tube needs no opening and the temperature is raised to melt the sealing layer so as to release the fluorescent dye, then the reaction tube is oscillated to mix the nucleic acid amplification product sufficiently with the fluorescent dye to react for a proper period of time, then the excitation light source irradiate the reaction tube, the fluorescence produced situation of the reaction tube can be observed via the observing window so as to analyze whether the sample to be detected has pathogenic microorganisms, if fluorescence is produced, the sample to be detected has pathogenic microorganisms. The method can directly carry out the nucleic acid amplification reaction and fluorescence detection in the instrument without taking out the reaction tube.

### EXAMPLE 4

### Terminating the Reaction by Adopting a Temperature-Controlled Reaction Tube

This embodiment realizes termination of the reaction by controlling the release of the reaction terminators in the sealing layer via temperature controlling, mainly for terminations of constant-temperature reactions such as RCA, LAMP. This method can control the reaction with a simple thermostat and is used under quick inspection conditions such as pathogenic microorganisms, disease diagnosis and so on.

The reaction terminator such as ethylene diamine tetraacetic acid (EDTA) solution or other metal complexing agent and protein denaturing agent is added in the bottom part of the reaction tube. 10 ul paraffins (80#) is added on the top surface. The reaction tube is heated till the melting point of the paraffin to melt the paraffins, then the reaction tube is taken out to be cooled, let the paraffins solidifies again to form the sealing layer.

When the RCA or LAMP reaction is carried out, the reaction system is added on the paraffin sealing layer. After the constant-temperature reaction (60° C.) is finished, the reaction tube is heated till the melting point of the paraffin, as the density of the paraffin is less than that of water, the paraffin will float on the surface layer of the liquid after the paraffin is melt, the reaction system will be mixed with the reaction terminators below the paraffin sealing layer so as to terminate the reaction.

The paraffins will solidify again after the reaction tube is taken out to be cooled so as to separate the liquid surface from the air and avoid lag pollutions possibly caused by the reaction product.

### EXAMPLE 5

### Detecting the Product with a Temperature-Controlled Reaction Tube

This embodiment realizes that the amplification reaction and the product indication in the same tube by controlling the release of the product indicator in the sealing layer via temperature. The method is mainly for constant-temperature amplification reactions such as RCA, LAMP and can realize fast, simple and visual inspection of pathogenic microorganisms.

Product indicators such as SYB Green, Gold View are added in the bottom part of the reaction tube. 10 ul paraffins (64#) is added on the top surface. The reaction tube is heated to the melting point of the paraffin to melt the paraffins. Then the reaction tube is taken out to be cooled to the room temperature so as to solidify the paraffin again to form the sealing layer.

When RCA or LAMP reaction is carried out, the reaction system is added on the paraffin sealing layer. After the constant-temperature reaction (60° C.) is finished, the reaction tube is heated to the melting point of the paraffin, as the density of the paraffin is less than that of water, the paraffin will float on the surface of the liquid after the paraffin is melt, the reaction system will be mixed with the product indicators to produce observable changes by naked eyes, so as to realize the visual detection of the amplification product.

The detecting method designed in this embodiment on one hand avoids to inhibit the amplification reaction caused by adding product indicators into the reaction system, on the other hand realizes the reaction and detection in the same reaction tube without opening the reaction tube and avoids the lag pollutions of the products.

### EXAMPLE 6

### Realizing Hot Start of Ordinary Heat-Resistant Polymerase and Color Reaction by Adopting a Temperature-Controlled Reaction Tube

Referring to FIG. 6, this embodiment realizes inhibition of non-singular amplification reaction at a low temperature by controlling the release of key ingredients of the reaction system in the sealing layer II via temperatures, meanwhile controlling the release of the color reagent in the sealing layer I via a higher temperature, namely the hot start of ordinary heat-resistant polymerase and color reaction are realized in a single tube. This method is mainly for PCR reactions, and can realize the hot start PCR with ordinary heat-resistant polymerases which is lower price and carry out color detection to the PCR products.

Product indicators such as SYB Green, Gold View are added in the bottom part of the reaction tube 15 (namely reagent I). 10 ul paraffins (95#) (namely the sealing material I) is added on the top surface. The reaction tube is heated to the melting point of the paraffins to melt the paraffins. Then the reaction tube is taken out to be cooled to room temperature so as to solidify the paraffins again to form the sealing layer I. Key ingredients such as one or several of heat-resistant polymerase, magnesium ion, dNTP and so on (namely reagent II) are added on the sealing layer I. 10 ul paraffins II (85#) (namely the sealing material II) is added on the key ingredients, the temperature thereof is lower than the PCR denaturation temperature and is also lower than the melting point of the paraffins I of the sealing layer I. The reaction tube is heated to the melting point of the paraffins II to melt the paraffins II. The reaction tube is taken out to be cooled to room temperature so as to solidify the paraffins II again to form the sealing layer II.

Before the PCR reaction, the reaction systems excluding the key ingredients in the sealing layer is added on the paraffin sealing layer II, and the tube cap 16 is closed. When the reaction is carried out, firstly the tube is heated above the melting temperature of the paraffin II and keeps constant temperature for 5 minutes so as to melt the paraffins sufficiently. As the density of the paraffin is less than that of water, the paraffins will float on the top layer of the liquid surface after the paraffins are melt, the reaction systems will mix with the reaction key ingredients (namely the reagent II) below the paraffins II, the temperature in the reaction tube accords with the temperature needed in the amplification reaction so as to start the amplification reaction.

When the amplification reaction is finished, the reaction tube is heated to the melting point of the paraffins I, as the density of the paraffin is less than that of water, the paraffins will float on the top layer of the liquid surface after the paraffins are melt, the reaction systems will mix with the product indicators (namely the reagent I); the temperature of the reaction tube accords with the temperature needed by the product indicators so observable changes by naked eyes are produced so as to realize the visual detection of the amplification product.

This method can carry out the hot start reaction with ordinary heat-resistant polymerases, inhibits the non-singular amplification reaction, enhances the specificity of the PCR reaction and meanwhile can carry out the color reaction to the amplification products in the same tube. (The reaction tubes of the two sealing layers are also for isothermal amplification reactions such as RCA, LAMP, all of part of the reagent needed in the nucleic acid amplification reaction are sealed in the sealing layer II (melting at a lower temperature), the fluorescent dye is sealed in the sealing layer I (melting at a higher temperature), sequential dissolving of the two sealing layers is realized by controlling the temperature changes to control the reaction process so as to carry out the nucleic acid amplification reaction and fluorescence visual detection.)

What is claimed is:

1. A method for fast detecting nucleic acid comprising:
adding a pathogenic microorganism nucleic acid into a temperature-controlled reaction tube, which comprises at least a sealing layer in which fluorescent dye is sealed, and carried out an amplification reaction by LAMP technology therein;
after the amplification reaction is completed raising temperature of the temperature-controlled reaction tube without opening the reaction tube to dissolve the sealing layer to release the fluorescent dye;
taking a fluorescent detection for amplification reaction.

2. The method according to claim 1, wherein said temperature-controlled reaction tube comprises first and second sealing layers, all or part of the reagents used in the nucleic acid amplification reaction are sealed in the first sealing layer which melts at a lower temperature, the fluorescent dye is sealed in the second sealing layer which melts at a higher temperature, the sequent dissolutions of two sealing layers are realized by changing temperature so as to realize the nucleic acid amplification reaction and fluorescence visual inspection in one tube without opening.

3. The method according to claim 1, wherein said fluorescent dye is SYBR Green 1 dye.

4. The method according to claim 1, wherein the amplification reaction and fluorescence detection are carried out directly in an instrument.

5. The method according to claim 1, wherein the fluorescence detection is realized by visual inspecting or photographing or analyzing images, data collected by a photoelectric sensor after the fluorescent dye produces fluorescence under excitation light.

6. A detecting device used in the detecting method according to claim 1 comprising:
a reaction tube oscillating device, a temperature adjusting device, a time adjusting device and a fluorescence color observing device respectively connected with a central control circuit; also can comprise a reaction tube lifting device connected with the central control circuit; also can comprise a temperature-controlled reaction tube with at least a sealing layer which melts at a proper temperature.

7. The detecting device according to claim 6, wherein the reaction tube oscillating device comprises a reaction tube rack, a rack oscillating motor which is connected with the reaction tube rack; the reaction tube lifting device comprises a drive motor for lifting the reaction tube rack and a reaction tube rack lifting rod, the drive motor for lifting the reaction tube rack is connected with the reaction tube rack lifting rod, and the reaction tube rack lifting rod is connected with the reaction tube rack.

8. The detecting device according to claim 6, wherein the temperature control device comprises a temperature control module, a heater and a temperature measuring device; the heater and the temperature measuring device are respectively connected with the temperature control module, the temperature control module is connected with the central control circuit or directly forms one part of the central control circuit, the temperature sensor of the temperature measuring device is near to the lower part of the reaction tube or near to the heater; the temperature adjusting device also can comprise a radiator connected with the temperature control module.

9. The detecting device according to claim 6, wherein the fluorescence color observing device comprises a fluorescence excitation light source and a fluorescence color observing or collecting device; the fluorescence color observing device is an observing window with or without a filter; the fluorescence color collecting device is an image collecting device or a photovoltaic conversion data collecting and analyzing device; the fluorescence excitation light source gives out light and irradiates the reaction solution in the reaction tube, and the fluorescence color observing or collecting device can observe or collect the fluorescence signal from the reaction solution in the reaction tube.

10. The detecting device according to claim 6, wherein the central control circuit further comprises a control information input or stored program control reaction type selecting device.
